(19) United States
(12) Patent Application Publication
(10) Pub. No.: US 2022/0313854 A1
(43) Pub. Date: Oct. 6, 2022

(54) APPARATUS CONFIGURED TO STORE AND SANITIZE OBJECTS

(71) Applicant: Sanitoyz, LLC, Van Nuys, CA (US)

(72) Inventor: Sarkis ARPAJIAN, Van Nuys, CA (US)

(21) Appl. No.: 17/711,585

(22) Filed: Apr. 1, 2022

Related U.S. Application Data
(60) Provisional application No. 63/170,219, filed on Apr. 2, 2021.

(51) Int. Cl. A61L 2/22; A61L 2/24

(52) U.S. Cl. CPC A61L 2/22; A61L 2/24; A61L 2202/11; A61L 2202/122; A61L 2202/14; A61L 2202/15

(57) ABSTRACT

A storage container system includes a storage unit including one or more shelves and two or more walls, an electrostatic sprayer system that includes a reservoir to store liquids, a charging unit, one or more nozzles configured to spray electrostatically charged liquid onto one or more objects located proximate the storage unit, wherein the nozzles are located on the one or more shelves or the two or more walls, and a controller in communication with the electrostatic sprayer system, wherein the controller is configured to send a first signal to initiate a spraying operation for a spraying threshold time in response to an activation signal.

### CROSS-REFERENCE TO RELATED APPLICATIONS

This application claims the benefit and priority of U.S. Provisional Application No. 63/170,219 filed Apr. 2, 2021, the disclosure of which is incorporated by reference herein.

### TECHNICAL FIELD

The embodiments described below related to storage systems and containers, including those with the ability to sanitize objects within the storage containers.

### BACKGROUND

Storage containers may be utilized to provide efficient storage of toys, tools, garments, and other objects. While many storage containers in the past contain efficient storage mechanisms, such systems may not have the capability to properly sanitize the objects stored within them. Items and objects that are handled frequently by people may need more regular and frequent cleaning. However, it may tedious and time-consuming to frequently clean such items and objects individually.

### SUMMARY

In a first embodiment, a storage container system includes a storage unit including one or more shelves and two or more walls, an electrostatic sprayer system that includes a reservoir to store liquids, a charging unit, one or more nozzles configured to spray electrostatically charged liquid onto one or more objects located proximate the storage unit, wherein the nozzles are located on the one or more shelves or the two or more walls, and a controller in communication with the electrostatic sprayer system, wherein the controller is configured to send a first signal to initiate a spraying operation for a spraying threshold time in response to an activation signal.

In a second embodiment, a storage container system includes a storage unit including one or more shelves and a door providing access to the one or more shelves, an electrostatic sprayer system that includes a reservoir to store liquids, a charging unit, one or more nozzles configured to spray electrostatically charged liquid onto one or more objects located proximate the storage unit, wherein the nozzles are located on the one or more shelves or the two or more walls, and a controller in communication with the electrostatic sprayer system, wherein the controller is configured to send a first signal to initiate a spraying operation for a spraying threshold time in response to an activation signal.

In a third embodiment, a storage container system includes a storage unit including one or more shelves and a door providing access to the one or more shelves, an electrostatic sprayer system that includes a charging unit and one or more nozzles configured to spray electrostatically charged liquid onto one or more objects located proximate the storage unit, wherein the nozzles are located on the one or more shelves or the two or more walls, and a controller in communication with the electrostatic sprayer system, wherein the controller is configured to send a first signal to initiate a spraying operation for a spraying threshold time in response to an activation signal.

### BRIEF DESCRIPTION OF THE DRAWINGS

FIG. 1 is an embodiment of a storage container from a front perspective view.

FIG. 2 is an example of a schematic diagram of the sanitizing system of the storage unit.

FIG. 3 is an embodiment of a storage container from a front perspective view opened up.

FIG. 4 is an example of a mobile device user interface utilized to operate and control the storage unit.

FIG. 5 is an example of the sanitizing system of the storage unit.

FIG. 6 discloses a method according to an example of operation of the electrostatic sprayer system is illustrated in a flowchart.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

Example embodiments of an storage unit with an electrostatic cleaning system constructed in accordance with the present disclosure will now be more fully described. Each of these example embodiments are provided so that this disclosure is thorough and fully conveys the scope of the concepts, features and advantages to those skilled in the art. To this end, numerous specific details are set forth such as examples of specific components, devices and mechanisms associated with the electrostatic cleaning system to provide a thorough understanding of each of the embodiments associated with the present disclosure. However, as will be apparent to those skilled in the art, not all specific details described herein need to be employed, the example embodiments may be embodied in many different forms, and thus should not be construed or interpreted to limit the scope of the disclosure.

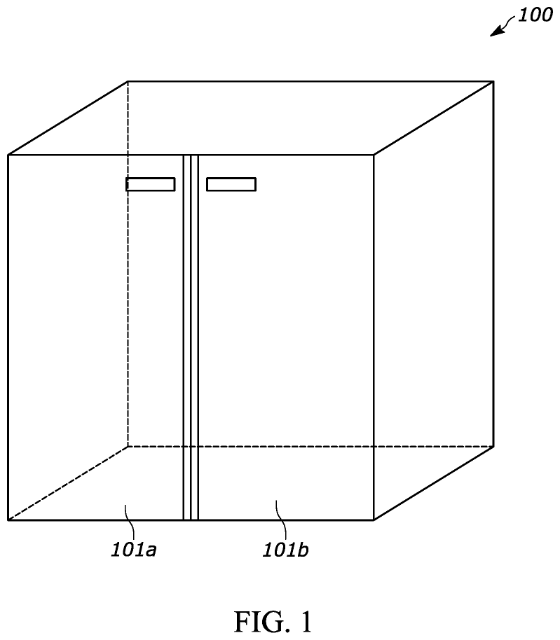

FIG. 1 is an embodiment of a storage container from a front perspective view. The storage unit may include one or more doors. In one embodiment, the storage unit may include two doors that swing open on hinges. In an alternative embodiment, the doors may slide open. In other embodiments, the doors may be a clear plastic to view the cleaning process or the objects that are contained therein. The doors may include a locking mechanism that prevent the doors from opening and therefore securing access to the items in the storage container. The storage container may include blow molded material or injected molded material in one embodiment. Material may also be made of metal or a hybrid between metal and plastics. The outer shell of the storage container may include a cavity that allows bottle containing a liquid that is utilized to sanitize various items. The liquid may be in a bottle that contains an opening that when engaged with the cavity at the outer shell, allows for the liquid to pass through the bottle to a sanitizing system of the storage unit. However, if the bottle is not engaged with the cavity, the bottle may be locked to prevent liquid from spilling through. In such a system, a closure or cap of the bottle is brought into engagement with a bottle piercing assembly, which is movably mounted in a socket. The bottle piercing assembly may include one or more piercing needles which puncture a portion of a cap of the bottle, such as an elastomeric gasket. One of the needles may be connected with a vent valve, which allows air to enter a reservoir as the cleaning liquid is dispensed. The other needle is fluidly connected with a pump. The gasket may be injection molded of silicone rubber. The solution may be inserted on bottom of the box or front with an interlocking twist which will fill the reservoir. The solution may be 180 ppm HOCL (hypochlorous acid) or more to sanitize the objects. Of course, any type of solution may be utilized. The box may be approximately 36″ wide, 36″ high, and 17″ deep, or any dimension.

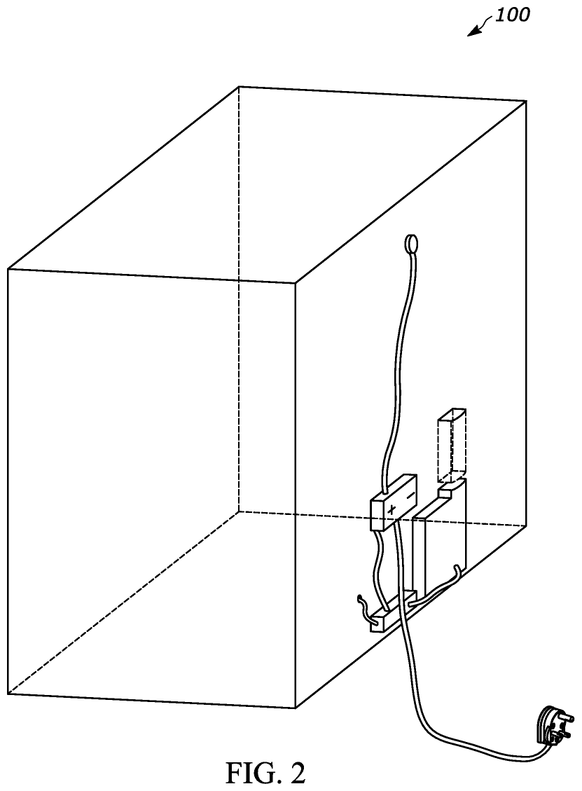

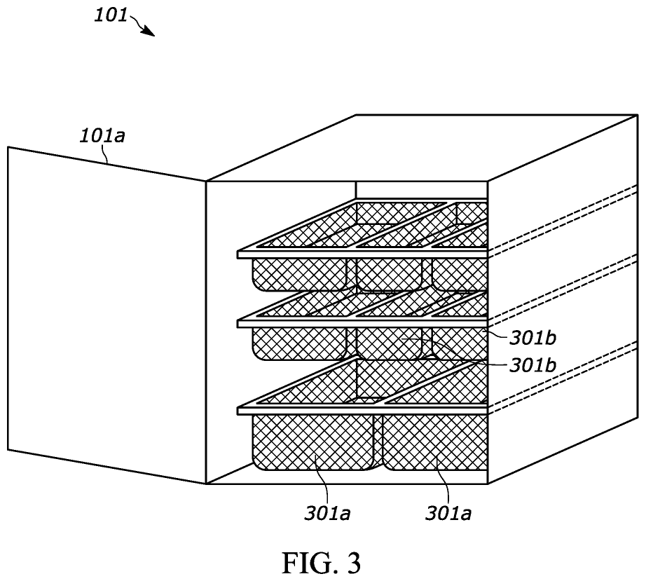

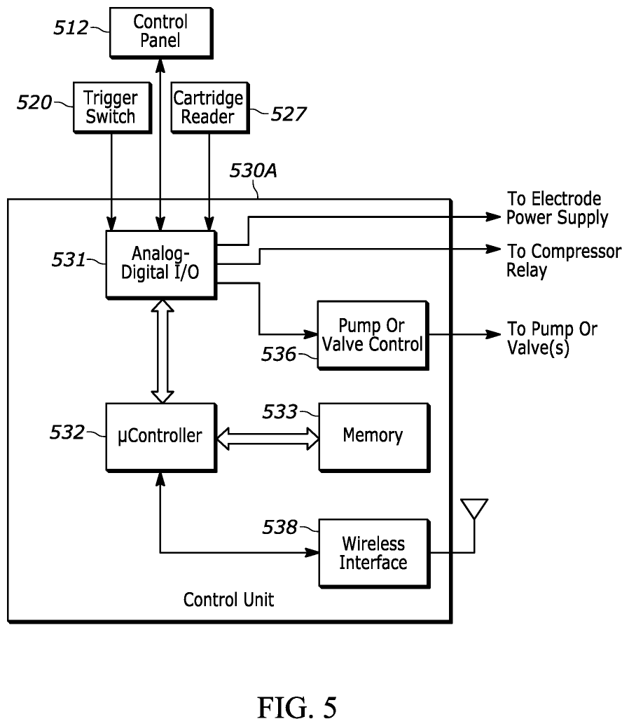

FIG. 2 is an example of a side profile of the sanitizing system of the storage unit 100 that emphasizes the various components of the sanitizing system, such as those disclosed in FIG. 5. The power may plug into an outlet. The power may be connected to a printed circuit board. Solution may be sucked in from a pump to a heat exchanger. The nozzle may include a metal inside that is connected to an ion generator. The mist may be a cooler steam rather than steam. In an embodiment, the mister or sprayer may be at the bottom of the box, which allows the heat to rise and make contact with all the FIG. 3 is an embodiment of a storage container 100 from a front perspective view opened up. The storage container may include one or more shelves. In one embodiment, the storage container 100 will have three shelves that have removable plastic/mesh bins. The shelves may be adjustable in terms of a height to allow for different dimensions of objects to be stored. The shelves may be level or may be slanted to allow for run-off in certain situations, such as run-off from various liquids. The shelves may also be pull out frames, similar to drawers. A back surface of the back panel of the storage container unit may include either an indent that the bin rests on top of, or an outdent that the bin rests on top of. Thus in various embodiments, the back surface may either have a protrusion for resting the bins, or an indent to rest the bins. Of course, any type of holding mechanism may work.

Containers may be included that are mesh that allow for liquids to run through. In another embodiment, the containers may be solid plastic containers. The containers may be any size. The containers may be stored on the shelves and also allow multiple different objects to be stored in the containers and to be secure in storage. The bins may rested on a slant with the back sitting on an indent of the mold or out dent. The front of the bins of each row will sit on a rod that is inserted in from the front via a C-channel setting and clicks in to a hole to lock in place when aligned. There may be another hole in the back of each row for the rods to slide back giving room to put larger toys.

In one embodiment, there may be two different bin sizes, although a multitude of different bin sized may exist. A first bin size may be 14″ deep, 5″ height, and 8″ width. A larger bin may be 16″ wide in another embodiment, however, any dimension may suffice.

The doors may be clear doors in one embodiment. In another embodiment, the doors may be the same material as the rest of the storage unit. The options of the doors may be any type of door, such as a swing door, a sliding door, a single door, a French-door, etc. The doors may include a magnetic seal strip. The magnetic seal may activate to lock the door, or be deactivated to allow the door to be easily opened. Additionally, the doors may include one or more middle dividers, flipper, French door, divider, etc. The doors may be configured to open outward, and close with a mullion that will seal and lock it. For example, in one embodiment, there may be a magnetic seal that is utilized to hold the doors tight.(possibly with a magnetic seal like a refrigerator as well). The doors may be equipped with an automatic locking mechanism/solenoid when the machine is running and spraying and/or drying, or preparing for those situations.

The bottom level of the shelf of the storage unit may include a basin or run-off to allow any liquid that is sprayed to be collected. Thus, at a lowest elevation point of the shelve, a drainage basin or another drainage area me be connected to the lowest elevation point in order to collect any liquid that is run off. The drainage area or basin may include a pan to collect the run off and dispose of it. In another embodiment, the drainage basin may include a removable plug that when removed, allows for the liquid to flow through and be emptied from the storage container.

The electrostatic sprayer may work by charging liquids (e.g., cleaners, sanitizers, and disinfectants) as they pass through a sprayer nozzle or another area of the sanitizing system. This may generate charged droplets that repel one another and actively seek out environmental surfaces, which they stick to and even wrap around to coat all sides. This may result in a uniform coating of sanitizer or disinfectant on sprayed objects, including hard-to-reach areas that manual cleaning can miss. The technology may helps avoid liquid pooling often associated with trigger sprayers or other sprayers.

The electrostatic sprayer system may require either a cordless or corded systems. Corded systems have inherent benefits that provide superior performance. For example, electrostatic sprayers using outlet power include an air compressor, which may reduce blowback by pushing sanitizer or disinfectant liquid toward environmental surfaces and away from the operator. Battery-powered sprayers may not contain an air compressor and more closely resemble trigger sprayers in terms of coverage and surface wrap.

The electrostatic sprayer system of the storage container may contain one or more hoses, a reservoir for storing liquid, a spraying end, and a charging unit (e.g., ion generator). The ion generator may be a negative or cation generator. The hose may be exposed in certain embodiments. In other embodiment, the hose may be built into panels along the interior walls of the storage container or walls of the shelves. Those may be utilized to allow liquid from the reservoir to ultimately travel to various areas of the storage unit. One of the exterior walls of the storage container may include an indented cavity to allow for insertion of the liquid solution into the reservoir tank. The electrostatic spraying works by charging liquids as they pass through a sprayer nozzle or other component. A tip of the nozzle may be adjusted to change a stream from a mist to a jet stream. The spray nozzles may be connected to the walls or shelves of the storage container and be adjusted to hit various areas within the storage container. There may be one or more misting nozzles that are utilized. The liquid ejected from the tip of the spray nozzle may coats surface more uniformly and generates a liquid cloud pattern that can reach hidden surfaces underneath and behind objects, providing more effective disinfection than would be possible with ordinary sprays and cleaning of the objects. The charging unit (e.g., ion generator or ionizer) may be inductive electrostatic, contact charging, or any type of charging, etc.

Once the cleaners, sanitizes, or disinfectants are charged and passed through the sprayer nozzle, this may generate charged droplets that repel one another and actively seek out environmental surfaces. They stick to, and even wrap around to coat all sides of surfaces. The result is a uniform coating of sanitizer or disinfectant on sprayed objects, including hard-to-reach areas that traditional cleaning methods can easily miss. The electrostatic spraying may also help avoid liquids from pooling which is often the result of using trigger sprayers.

The electrostatic sprayer system may be activated by a power button on the storage container. The power button may be located on an exterior of the storage unit or in an interior wall of the storage container that is only visible when opened by the doors. The power button may include a connection to the electrostatic sprayer system to begin activation. The power button may activate the spraying for a threshold time (e.g. 3 minutes), as well as locking the doors via a locking mechanism until the threshold time expires. Upon the threshold time expiring, the system may include a threshold drying period. The system may unlock the doors after a drying period is up.

The storage container may also include a controller or processor that is utilized for programming various commands. Such commands may include turning the electrostatic sprayer system on or off. Another command may include automated locking and unlocking of the doors, or opening and closing of the doors. The controller may also include memory and a timer or clock to prepare a schedule for utilization.

In one embodiment, the storage container may include a wireless transceiver connected to the processor and in communication with a mobile phone and/or an off-board server. The wireless transceiver may be utilized for communicating data to the mobile phone or off-board server. The wireless transceiver may be a Bluetooth transceiver, cellular modem, Wi-Fi modem, etc. In one embodiment, the wireless transceiver may be in communication with a mobile device that includes an application utilized to control various settings.

The storage container may also include a hardwire plug to plug in for electricity or a battery pack. The plug may allow a charging unit to charge the liquid in the reservoir. The storage container may operate utilizing a pump that pumps the liquid through the hose and out of an outlet of the sprayer end. The power supply (either plug or battery pack) may provide an output of electricity coupled to the electrode wire to an electrode that may charge the liquid within the reservoir, hose, or tip of a sprayer. The electrode wire may be found in the reservoir, the nozzle end, and/or other areas of the storage unit. The electrode wire may provide electrical current to an electrode through a contactor. A socket may receive the contactor and is electrically connected to electrode wire. With such an arrangement, both nozzle electrode wire and a liquid hose may be protectively encased in a mounting tube. Mounting tube may be utilized to protect the electrode wire to eliminate exposure to the wet environment. In one embodiment, mounting tube may be manufactured from an electrically-insulating material to offer further protection against leakage currents from the sources of high voltage to the liquid stream or earthed components. In one embodiment, an electrically-insulating dielectric shroud at least partially surrounds a base portion of liquid tip and in another embodiment, is press-fit into the nozzle body. In one embodiment, a dielectric shroud may be integral to nozzle body, or may be made removable for cleaning or replacement, by fitting dielectric shroud into a press-fit recess in nozzle body, or by a threaded connection. Dielectric shroud may be fabricated from the same material as nozzle body, or of a different material.

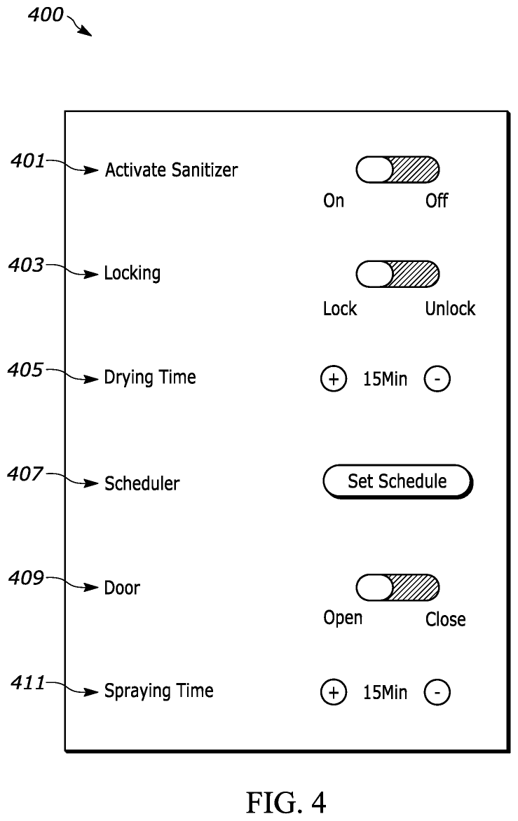

FIG. 4 is an example of an interface 400 utilized to operate and control the storage unit. The interface 400 may be a display on the storage unit or a mobile device interface. The mobile device may communicate with the storage unit to operate various functions and to communicate data from the storage unit to remote devices, such as the mobile devices. The storage unit may communicate to the mobile device via a wifi interface or utilizing a Bluetooth interface. The mobile device can thus send a signal remotely to the storage unit to either activate on or activate off. Another function may include locking the doors or unlocking the doors. The application may also allow a schedule to be activated or set for the storage unit via a scheduler option 407. The scheduler 407 may allow the storage unit to activate the sanitizing function on certain times (e.g., 6:30 PM); certain dates (e.g., every Monday, Wednesday, and/or Friday), and to repeat on certain times. Another setting of the application may allow for adjustment of the time threshold for spraying 411 within the storage unit, as well as the drying period 405. The application may also be useful for providing notifications upon a complete cleaning cycle, a drying cycle, maintenance warnings (e.g., low liquid levels, descaling, identify disinfectants, and other maintenance). The application may also exchange data from the storage unit to the mobile device, such as the counter, reservoir level, door open/close status, etc. The processor may communicate with various sensors in the storage unit that are then in communication with the controller. From there, the controller may be in communication with a wireless transceiver that sends such information directly to the mobile device or to the mobile device via the cloud (e.g., remote server).

FIG. 5 is an example of the sanitizing system of the storage unit. Referring now to FIG. 5, details of a control unit 530A for the example electrostatic sprayer are shown. A microcontroller 532 (e.g., or processor, controller, etc.) receives inputs and controls operation of the electrostatic sprayer via a program, i.e., a computer program product, stored in a memory 533, which also stores data. An analog/digital I/O unit 531 receives signals from internal circuits of the electrostatic sprayer and generates control output signals according to the program contained in memory 533, which forms a computer program product having unique features as described herein. Communications may be sent to, and control input received from, a remote computer system via a wireless interface 538. Wireless interface 538 may be a 802.11 “wifi” interface, a Bluetooth Interface, a Near-Field interface, an audio link or an optical link such as an infrared (IR) link. Wireless interface 538 may link directly with the remote computer, or may interact with an application or systems' function on a wireless device, such as a tablet or mobile telephone used by the user in conjunction with the electrostatic sprayer to perform control functions. For example, wireless interface 538 may be a Bluetooth® interface that communicates with an application executing within a mobile telephone of the user to control the sprayer. Additionally, the wife interface 538 may be utilized to communicate data about the contents inside of the storage container (e.g., if a camera is located inside of the bin), to The system may also utilize a heat sink in some embodiments to transfer heat generated from the various components of the system to the liquid and dissipate away from the components. The heat exchanger may allow for heat from a fluid (a liquid or a gas) to pass to a second fluid (another liquid or gas) without the two fluids having to mix together or come into direct contact. The heat exchanger may transfer the heat without transferring the fluid that carries the heat. In one embodiment, the heat exchanger may not be present or may not be utilized. Thus, the sanitizer of fluid from the fluid reservoir may be expected to be an ambient temperature in such embodiments. However, a heated fluid may allow for a better disinfecting of fluid and to allow for more of a mist than unheated fluid. Between the pump and the ionizer may be a heat exchange. The heat exchange may be utilized to heat the solution that is in the reservoir. The heat exchange may heat the solution to a number of different temperatures.

Microcontroller 532 is also interfaced to control panel 518 to provide functions as described herein. Analog-Digital I/O 531 further provides signals to a pump motor or valve control 536 and/or air compressor relay. Analog-Digital I/O 531 also provides input power for electrode power supply which generally will be located close to electrode. Analog-Digital I/O 531 includes analog-to-digital converters (ADCs) and digital-to-analog converters (DACs) as required to convert analog signals to digital information and vice-versa. Analog-Digital I/O 531 may be provided by converters integrated within microcontroller 532. Analog-Digital I/O 531 receives a feedback signal from electrode power supply indicating the current drawn by electrode, which informs microcontroller 532 of the level of the current. As noted above, the voltage and current level generally need to be controlled at different levels for the spraying of the active agent and any spraying of a non-active liquid.

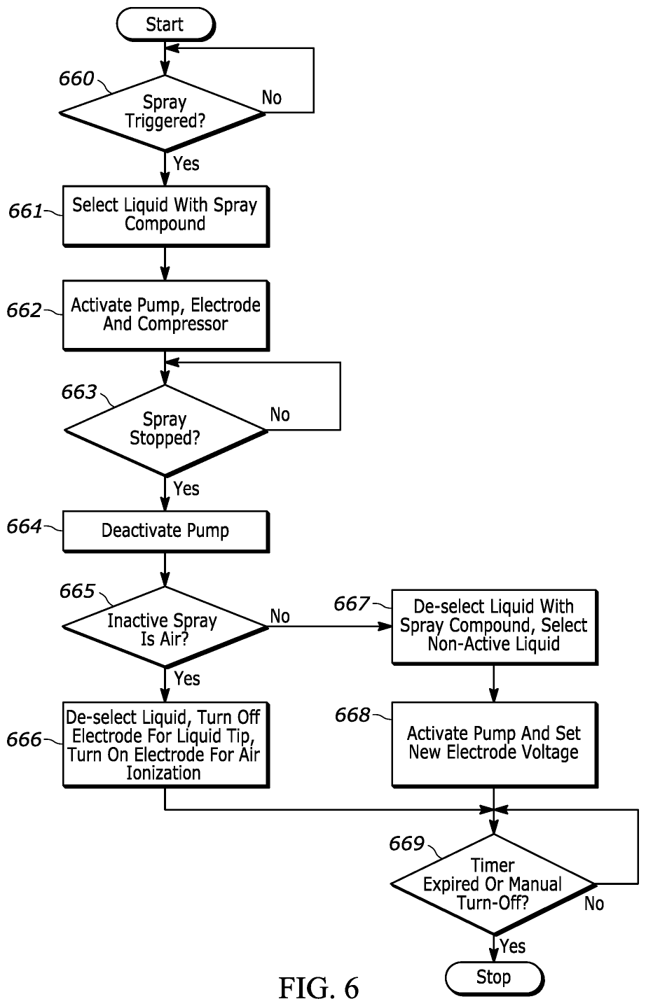

Referring now to FIG. 6, a method according to an example of operation of the electrostatic sprayer system is illustrated in a flowchart. When spraying is triggered/activated (decision 660), the liquid containing the active agent is selected for spraying (step 661) and the valve (or pump) that controls the flow of the active liquid and the electrode that charges the liquid spray are activated (step 662). When spraying of the active liquid is stopped (decision 663) (or alternatively a selection is made to disperse the residual spray cloud), the valve or pump activated in step 662 is de-activated (step 664). If the non-active spray is to be ionized air (decision 665), then the active liquid is de-selected, the electrode is turned off, and the electrode for air ionization is activated (step 666). If a non-active liquid is used to disperse the spray cloud, then the non-active liquid is selected (step 667) the pump is activated and a new electrode voltage is set for spraying the non-active liquid (step 668). Until a timer expires or the spraying of the inactive liquid or ionized air is turned-off by manual command (decision 669), spraying of the inactive liquid or ionized air continues. As mentioned above, steps 667 and 668 can be commenced before stopping the spraying of the active agent in some embodiments that support overlapped spraying of the active liquid and the inactive liquid or ionized air, in which case step 664 and the de-selection of active liquid will be performed later after a predetermined interval.

In one embodiment, the user may activate a start button on a switch, button, or display (e.g., storage container display, mobile device, computer, etc.). After activation, the doors may lock and the heater may begin heating the liquid. However, the heating may be optional. There may be a wait of 15-30 seconds to reach the needed temperature of the fluid, which may fluctuate or even be ambient temperature. A temperature sensor may be utilized to measure the temperature of the fluid. Upon the temperature of the fluid reaching the threshold temperature (or surpassing it), the pump may be activated to disperse and mist the solution through the nozzle. The nozzle may then spray the solution for a time period that is set at the factory or by a user. The typical spray time for the solution may between 1-2 minutes, but of course any amount of time will be configurable. After spraying, the pump may turn off but the heater may turn on for any duration, such at 10-15 seconds. Next, the pump may be reengaged for a second mist. The second mist may be any duration, but it may be a shorter duration in one embodiment. For example, the second mist may be a time period of about 30 seconds to 1 minute. After the second misting period, the pump and heater may turn off. Then, the storage container may stay locked for 10-15 minutes to allow for drying. In another embodiment, a fan may work with the heat exchange to blow hot air to increase the drying process. Upon completion of the process, the storage container may be unlocked. In another embodiment, a sounded signal may alert from a speaker of the storage container unit that notifies a user that cycle is done and it can be opened. In another embodiment, a wireless signal may be sent to a mobile device or remote computer to activate an alert, notification, or sound.

The system may also include the option to notify a user when the cleaning solution is low and to send a message to purchase the cleaning solution. A sensor may be utilized that identifies that the cleaning solution is low. One example may include a weight sensor that measures the weight of the solution, and when a solution is low, it is a certain weight. Thus, the system may determine if a threshold hold weight has been reached or passed as being lower than required for sufficient solution. In another embodiment, a moisture sensor may be utilized that is at a certain portion of the reservoir or within another portion of the storage unit (e.g., hose). The moisture sensor may be place at a spot to identify that there is no moisture, thus the solution is low. Thus, when the moisture sensor's reading falls below a certain threshold, the system may determine no moisture is present and thus the solution must be low. While a moisture sensor and a weight sensor are two examples, any type of sensor may be utilized to identify that the solution is low. The system may either output an alert at a display of the storage unit, a mobile phone in connection with the storage unit, or send another alert or email associated with the storage unit. The alert may indicate that the solution is low and provide options to purchase the solution from a reputable store or website. The system may also include the option to notify a user when maintenance is required, such as descaling of the storage unit. The storage unit may include a sensor to identify the number of cycles that our ran, the amount of time that the unit has been in operation, or any other type of maintenance indicators.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A storage container system, comprising:
a storage unit including one or more shelves and two or more walls;
an electrostatic sprayer system that includes a reservoir to store liquids, a charging unit, one or more nozzles configured to spray electrostatically charged liquid onto one or more objects located proximate the storage unit, wherein the nozzles are located on the one or more shelves or the two or more walls; and
a controller in communication with the electrostatic sprayer system, wherein the controller is configured to send a first signal to initiate a spraying operation for a spraying threshold time in response to an activation signal.

2. The storage container system of claim 1, wherein the controller is configured to send a second signal to initiate a drying operation upon completion of the spraying operation, wherein the drying operation is a passive drying or an active drying.

3. The storage container system of claim 2, wherein the second signal further initiates a locking function of the storage unit for a locking threshold time period, wherein the locking function locks one or more doors of the storage unit.

4. The storage container system of claim 1, wherein the storage unit includes one or more wireless transceivers configured to communicate data from the storage unit.

5. The storage container system of claim 1, wherein the one or more shelves are removable from the storage unit.

6. A storage container system, comprising:
a storage unit including one or more shelves and a door providing access to the one or more shelves;
an electrostatic sprayer system that includes a reservoir to store liquids, a charging unit, one or more nozzles configured to spray electrostatically charged liquid onto one or more objects located proximate the storage unit, wherein the nozzles are located on the one or more shelves or the two or more walls; and
a controller in communication with the electrostatic sprayer system, wherein the controller is configured to send a first signal to initiate a spraying operation in response to an activation signal.

7. The storage container system of claim 6, wherein the controller is configured to send a second signal to initiate a drying operation upon completion of the spraying operation, wherein the drying operation is a passive drying or an active drying.

8. The storage container system of claim 7, wherein the second signal further initiates a locking function of the door for a locking threshold time period.

9. A storage container system, comprising:
a storage unit including one or more shelves and a door providing access to the one or more shelves;
an electrostatic sprayer system that includes a charging unit and one or more nozzles configured to spray electrostatically charged liquid onto one or more objects located proximate the storage unit, wherein the nozzles are located on the one or more shelves or the two or more walls; and
a controller in communication with the electrostatic sprayer system, wherein the controller is configured to send a first signal to initiate a spraying operation for a spraying threshold time in response to an activation signal.

10. The storage container system of claim 9, wherein the controller is configured to send a second signal to initiate a drying operation upon completion of the spraying operation, wherein the drying operation is a passive drying or an active drying.

11. The storage container system of claim 10, wherein the second signal further initiates a locking function of the door for a locking threshold time period.

12. The storage container system of claim 9, wherein the electrostatic sprayer system includes a reservoir to store liquids.

13. The storage container system of claim 9, wherein the electrostatic sprayer system includes a heat exchange configured to heat liquid.

14. The storage container system of claim 9, wherein the electrostatic sprayer system is configured to initiate spraying of electrostatically charged liquid in response to activation on either the storage container system or a display of a mobile device in communication with the storage container system.

15. The storage container system of claim 9, wherein the system includes a wireless transceiver configured to send data to a mobile device, wherein the data includes information associated with the storage container system.

16. The storage container system of claim 15, wherein the data includes information associated with activation of sanitizing, locking of the storage container system, a drying time, a schedule, opening a door, closing a door, and a spraying time.

17. The storage container system of claim 15, wherein the wireless transceiver is a Wi-Fi transceiver.

18. The storage container system of claim 15, wherein the wireless transceiver is a Bluetooth transceiver.

19. The storage container system of claim 15, wherein the data includes information associated with a maintenance alert associated with the storage container system.

20. The storage container system of claim 15, wherein the data includes information associated with a low-solution warning associated with the storage container system.
